(19) United States
(12) Patent Application Publication
(10) Pub. No.: US 2019/0201298 A1
(43) Pub. Date: Jul. 4, 2019

(54) PRODUCT AND METHOD FOR LOCAL CLEANSING, BEAUTIFYING, PROMOTING ATTRACTIVENESS OR ALTERING APPEARANCE OF A SUBJECT

(71) Applicant: Rocketfuelabs, LLC, New York, NY (US)

(72) Inventor: Bernardino Reynoso, Brooklyn, NY (US)

(21) Appl. No.: 16/238,267

(22) Filed: Jan. 2, 2019

Related U.S. Application Data
(60) Provisional application No. 62/613,226, filed on Jan. 3, 2018.

(51) Int. Cl. A61K 8/02; A61Q 19/00; A45D 44/00

(52) U.S. Cl. CPC A61K 8/0212; A61Q 19/007; A45D 44/22; A61K 8/0208; A45D 44/002

(57) ABSTRACT

The described invention relates, in part, to cosmetic sheet structures for application to a region of skin of the human body, the cosmetic sheet structure comprising a cosmetic layer comprising material impregnated with at least active ingredient contained in the cosmetic sheet structure and transferrable to the region. According to some embodiments, the cosmetic sheet structures are used to improve the appearance of skin and/or for decorative purposes.

### RELATED APPLICATIONS

This application claims priority to U.S. Provisional Application No. 62/613,226, filed on Jan. 3, 2018, the contents of which are incorporated by reference in their entirety herein.

### BACKGROUND

The skin, which is the largest organ in the human body, typically requires cosmetic care to maintain it in good condition. Personal care products that are administered topically are generally known as topical agents. One concern when administering topical agents is the ease with which these agents can be applied to the skin. Usually, the topical agent is dispensed from a bottle or tube onto the fingers or palm of a hand, and applied manually to the skin. A consumer is disadvantageously required to carry the bottle or tube on their person when they travel. However, particular skin care products may not be available in a travel size. Moreover, such products are most often available as a liquid, cream or ointment, and can be messy, and it can be hard to target a particular area of interest.

People are continually looking for a product that can improve the visual appearance of their skin. For instance, symptoms associated with dry skin (e.g., flaky skin, dried or rough tactile quality, cracked skin, dehydrated skin, itchy skin, or red or erythemic skin) or symptoms associated with aging skin (age spots, dryness, fine lines and wrinkles) are associated with unattractive skin. Previous attempts to create a composition to treat or prevent dry skin have been meet with numerous drawbacks ranging from compositions having unpleasant tactile properties (e.g., heavy, greasy, or sticky feel), low staying power (e.g., tendency to migrate and pool from point of application, or tendency to be easily removed from the skin via being subjected to only one washing), or insufficient moisturization or UV protection capabilities.

Emerging and/or poor market segments have additional concerns. For example, clean water may not be readily available, and waste can have a significant environmental impact.

Accordingly, it is an object of the described invention to provide a delivery system for active ingredients, for example cosmetic ingredients or pharmaceutical ingredients, that requires minimal water, minimizes packaging, and produces minimal waste. Another object of the described invention is to provide a delivery system for active ingredients which is at least as effective as liquid carriers in eliminating the signs of aging, especially treatment of age spots, dryness, fine lines and wrinkles.

These and other features and advantages of the described invention will become more readily apparent from consideration of the following summary and detailed description.

### SUMMARY OF THE INVENTION

The described invention features cosmetic sheet structures for application to the skin that that require minimal water, that minimizes packaging and produce minimal waste. In a first aspect, the described invention features a cosmetic sheet structure for application to a region of skin of the human body, the cosmetic sheet structure comprising a cosmetic layer comprising material impregnated with at least one active ingredient contained in the cosmetic sheet structure and transferrable to the region. According to some embodiments, the cosmetic sheet structure further comprises a backing. According to some embodiments, the active ingredient is a cosmetic ingredient or a pharmaceutical ingredient. According to some embodiments, the cosmetic sheet structure is applied to the face and/or neck. According to some embodiments the cosmetic sheet structure is applied to a part of the body, excluding the face. According to some embodiments, the skin is aged skin. In some embodiments, the skin is dry skin. According to some embodiments, the at least one active ingredient is formulated to form a film on the skin. According to some embodiments, the film is protective. According to some embodiments, the film is occlusive. According to some embodiments, the active ingredient is selected from the group consisting of antioxidants, free-radical scavengers, moisturizers, depigmenting agents, liporegulators, anti-acne agents, anti-aging agents, cleansers, softeners, anti-wrinkle agents, keratolytic agents, anti-inflammatories, refreshening agents, cicatrizing agents, tanning agents, vascular protective agents, antibacterials, antifungals, antiperspirants, deodorants, antidandruff agents, skin conditioners, immunomodulators, and nourishing agents. According to some embodiment, the active ingredient is selected from the group consisting of: blush, bronzer, eyeshadow, lip pigment and concealer. According to some embodiments, the active ingredient is transferred to the region when the cosmetic sheet structure is heated by touch. According to According to some embodiments, the active ingredient is transferred to the region when the cosmetic sheet structure is wetted by water or an aqueous solution. According to some embodiments, the active ingredient is effective to penetrate the superficial layers of the skin. According to some embodiments, the backing is attached to the cosmetic layer such that the matrix is capable of being separated from the cosmetic layer. According to some embodiments, the backing is attached to the cosmetic layer by moisture. According to some embodiments, the cosmetic sheet structure is flexible. According to some embodiments, the cosmetic sheet structure is transferrable from one region of the skin to another region of the skin. According to some embodiments, the cosmetic sheet structure forms a tape for application to the skin. According to some embodiments, the cosmetic sheet structure forms a shape for application to the skin. According to a related embodiment, exemplary shapes include, without limitation, square (with or without rounded edges), rectangular (with or without rounded edges), circular, oval, elliptical, kidney shaped, diamond, heart, or star. According to some embodiments, the shape is formed to match the area where it is applied. According to some embodiments, the shape is a customized shape. According to some embodiments, application of the shape to the skin is decorative. According to some embodiments, the thickness of the cosmetic sheet structure can be at least 0.01 cm, at least 0.02 cm, 0.03 cm, 0.04 cm, 0.05 cm, 0.06 cm, 0.07 cm, 0.08 cm, 0.09 cm, 0.1 cm, 0.2 cm, 0.3 cm, 0.4 cm, 0.5 cm, 0.6 cm, 0.7 cm, 0.8 cm, 0.9 cm, or 1 cm. According to some embodiments, the cosmetic sheet further comprises one or more ingredients selected from the group consisting of whitening agents, ultraviolet absorbents, surfactants, humectants, thickeners, alcohols, powder ingredients, coloring agents, pH adjusting agents, stabilizers, preservatives, perfumes, water, glitter, and various skin nutrients. According to some embodiments, the cosmetic sheet structure comprises a non-woven matrix. According to some embodiments, the cosmetic sheet structure comprises a nanofiber web obtained by electrospinning a water-soluble polymer and the cosmetic ingredient together. According to some embodiments, the cosmetic sheet structure is hypoallergenic. According to some embodiments, the cosmetic sheet structure is applied to the region of skin at least one a day. According to some embodiments, the cosmetic sheet structure is applied to the region of skin at least two or more times a day. According to some embodiments, the cosmetic sheet structure is packaged in a protective cover. According to some embodiments, the cosmetic sheet structure is contained within a dispensing unit. According to a further related embodiment, the dispensing unit comprises a jagged edge to tear the sheet. According to some embodiments, a second dispensing unit comprises a second cosmetic sheet structure. According to some embodiments, the dispensing units are connected. According to a related embodiment, the dispensing units are connected by one or more magnets. According to some embodiments, the one or more cosmetic sheet structures are contained within a housing unit. According to some embodiments, the housing unit further comprises a mirror. According to some embodiments, the one or more cosmetic sheet structures are contained within one or more housing units. According to According to some embodiments, the housing units are connected. According to some embodiments, the housing units are connected by one or more magnets. According to some embodiments, the cosmetic sheet dissolves after activation. According to some embodiments, the cosmetic sheet does not leave a residue on the area of the skin after dissolving. According to some embodiments, once the product is applied and activated with very little water, only then it dissolves to spread throughout the skin area leaving no residue.

According to another aspect, the described invention features a method of improving the appearance of the skin, the method comprising contacting one or more regions of the skin with one or more cosmetic sheet structures as described in any of the aspects and embodiments herein, thereby improving the appearance of the skin. According to some embodiments, an improvement in the appearance of the skin comprises an improvement in one or more of wrinkles, dryness, discoloration, imperfection, scarring, redness, radiance, softness or blemishing of the skin.

According to another aspect, the described invention features a method of protecting the skin from external damage, the method comprising contacting on or more regions of the skin with one or more cosmetic sheet structures as described in any of the aspects and embodiments herein, thereby protecting the skin from external damage. According to some embodiments, the external damage is caused by ultraviolet radiation. According to some embodiments, the external damage is caused by temperature.

According to another aspect, the described invention features a method of applying a cosmetic composition to the skin, the method comprising contacting one or more regions of the skin with one or more cosmetic sheet structures as described in any of the aspects and embodiments herein, thereby protecting the skin from external damage. According to some embodiments, the cosmetic composition comprises a pigment. According to some embodiments, the cosmetic composition is selected from the group consisting of blush, bronzer, eyeshadow, lip pigment and concealer. According to some embodiments of any one of the above aspects or embodiments, the one or more cosmetic sheet structures are applied to the same area of the skin. According to some embodiments of any one of the above aspects or embodiment, the one or more cosmetic sheet structures are applied to multiple different areas of the skin. According to some embodiments of any one of the above aspects or embodiments, the one or more cosmetic sheet structures are activated by water or an aqueous solution. According to some embodiments of any one of the above aspects or embodiments, the one or more cosmetic sheet structures are activated by heat. According to some embodiments of any one of the above aspects or embodiments, the one or more cosmetic sheets dissolve after activation. According to some embodiments, the cosmetic sheets dissolve about 30 seconds to 1 hour after activation. According to some embodiments, the cosmetic sheets do not leave a residue on the area of the skin after dissolving. According to some embodiments of any one of the above aspects or embodiments, the one or more cosmetic sheet structures are applied to multiple different areas of the skin. According to some embodiments of any one of the above aspects or embodiments, the one or more cosmetic sheet structures are applied for a time of about 1 minute to 5 hours. According to some embodiments of any one of the above aspects or embodiments, the one or more cosmetic sheet structures are applied at least once a day. According to some embodiments of any one of the above aspects or embodiments, the one or more cosmetic sheet structures are applied at least once a day. According to some embodiments of any one of the above aspects or embodiments, once the product is applied and activated with very little water, only then it dissolves spread throughout the skin area living no residue.

### BRIEF DESCRIPTION OF THE DRAWINGS

FIG. 1 is a schematic that shows an exemplary method for using the cosmetic sheet structures.

FIG. 2 is a schematic of a dispensing unit for the cosmetic sheet structures. The dispensing unit may include a jagged edge to tear each strip along the edge of the dispensing unit. Multiple dispensing units are used to carry different strips, wherein the units may snap to each other or attach to each other with magnets.

FIG. 3 is a schematic of an exemplary housing unit.

FIG. 4 is a schematic of a housing unit for face caviar pearls or pods.

FIG. 5 is a schematic that shows a seven-day face caviar dispenser.

### DETAILED DESCRIPTION

The described invention provides, in part, a cosmetic sheet structure for application to a region of skin of the human body, the cosmetic sheet structure comprising a cosmetic layer comprising material impregnated with at least active ingredient contained in the cosmetic sheet structure and transferrable to the region. The cosmetic sheet structures require minimal water, minimize packaging and produce minimal waste. The cosmetic sheet structures as described herein have a variety of different uses, for example in improving the appearance of the skin, protecting the skin, treating the skin, for decorative use, and for applying a cosmetic composition.

Further areas of applicability of the described invention will become apparent from the detailed description provided herein. It should be understood that the detailed description and specific examples, while indicating the preferred embodiment of the invention, are intended for purposes of illustration only and are not intended to limit the scope of the invention.

### I. Definitions

As used throughout, ranges are used as shorthand for describing each and every value that is within the range. Any value within the range can be selected as the terminus of the range. In addition, each reference cited herein is hereby incorporated by referenced in its entirety. In the event of a conflict in a definition in the described disclosure and that of a cited reference, the described disclosure controls.

Unless otherwise specified, all percentages and amounts expressed herein and elsewhere in the specification should be understood to refer to percentages by weight. The amounts given are based on the active weight of the material.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “substantially” and its variations are defined as being largely but not necessarily wholly what is specified as understood by one of ordinary skill in the art, and according to one non-limiting embodiment substantially refers to ranges within 10%, within 5%, within 1%, or within 0.5%.

The term “carrier” as used herein describes a material that does not cause significant irritation to an organism and does not abrogate the biological activity and properties of the active agent. Carriers must be of sufficiently high purity and of sufficiently low toxicity to render them suitable for administration to the subject. The carrier can be inert, or it can possess pharmaceutical benefits, cosmetic benefits or both. The terms “excipient”, “carrier”, or “vehicle” are used interchangeably to refer to carrier materials suitable for formulation and administration of the active agents described herein. The phrase “cosmetically acceptable carrier” as used herein refers to a substantially non-toxic carrier, conventionally useable for the topical administration of cosmetics, with which the active agents of the described invention will remain stable and bioavailable.

The term “contact” and its various grammatical forms as used herein refers to a state or condition of touching or of immediate or local proximity.

The term “cosmetic” as used herein refers to a component or ingredient for cleansing, beautifying, promoting attractiveness, or altering the appearance, or an article intended for use as a component of any such article, except that such term does not include soap.

The term “disease” or “disorder”, as used herein, refers to an impairment of health.

As used herein, the term “improve” refers to a change (e.g. a decrease or an increase) compared to a control or a change (e.g. a decrease or an increase) compared to a subject prior to administration of the cosmetic sheet structures of the described invention.

The term “effective,” as that term is used in the specification and/or claims, means adequate to accomplish a desired, expected, or intended result.

The phrase “improving the appearance of the skin” includes slowing or reversing clinical or esthetical symptoms of a condition, substantially ameliorating clinical or esthetical symptoms of a condition, substantially preventing the appearance of clinical or esthetical symptoms of a disease, condition, or disorder, protecting from harmful or annoying symptoms, and decorative uses. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s). Improving the appearance of skin can be an improvement in moisturization, smoothness or brightening. Improving the appearance of skin can be decreasing the number of wrinkles or fine lines, or decreasing the rough texture of the skin. Improving the appearance of skin can be a reduction in dryness of the skin.

The terms “increasing” or “increase” or any variation of these terms, when used in the claims and/or the specification includes any measurable increase to achieve a desired result.

The terms “inhibiting” or “reducing” or any variation of these terms, when used in the claims and/or the specification includes any measurable decrease or complete inhibition to achieve a desired result.

“Percutaneous absorption” is the absorption of substances from outside the skin to positions beneath the skin, including into the blood stream. The epidermis of human skin is highly relevant to absorption rates. Passage through the stratum corneum marks the rate-limiting step for percutaneous absorption. The major steps involved in percutaneous absorption of, for example, a drug, include the establishment of a concentration gradient, which provides a driving force for drug movement across the skin, the release of drug from the vehicle into the skin-partition coefficient and drug diffusion across the layers of the skin-diffusion coefficient. The relationship of these factors to one another is summarized by the following equation:

J=Cveh×Km·D/x [formula 1]

where J=rate of absorption

Cveh=concentration of drug in vehicle

Km=partition coefficient

D=diffusion coefficient.

There are many factors which affect the rate of percutaneous absorption of a substance. Primarily they are as follows: (i) Concentration. The more concentrated the substance, the greater the absorption rate; (ii) Size of skin surface area to which the drug is applied. The wider the contact area of the skin to which the substance is applied, the greater the absorption rate; (iii) Anatomical site of application. Skin varies in thickness in different areas of the body. A thicker and more intact stratum corneum decreases the rate of absorbency of a substance. The stratum corneum of the facial area is much thinner than, for example, the skin of the palms of the hands. The facial skin's construction and the thinness of the stratum corneum provide an area of the body that is optimized for percutaneous absorption to allow delivery of active agents both locally and systemically through the body; (iv) Hydration. Hydration (meaning increasing the water content of the skin) causes the stratum corneum to swell which increases permeability; (v) Skin temperature. Increased skin temperature increases permeability; and (vi) The composition of the active agent and of the vehicle also determines the absorbency of a substance. The protein portion of the stratum corneum is most permeable to water soluble substances and the liquid portion of the stratum corneum is most permeable to lipid soluble substances. It follows that substances having both liquid and aqueous solubility can traverse the stratum corneum more readily. See Dermal Exposure Assessment: Principles and Applications, EPA/600/8-91/011b, January 1992, Interim Report—Exposure Assessment Group, Office of Health and Environmental Assessment, U.S. Environmental Protection Agency, Washington, D.C. 20460.

The term “skin” as used herein refers to the membranous protective covering of the body consisting of the epidermis and corium (dermis).

All percentages, parts and ratios as used herein are by weight of the total dosage form, unless otherwise specified. All such weights as they pertain to listed ingredients are based on the active level and, therefore do not include solvents or by-products that may be included in commercially available materials, unless otherwise specified.

### II. Cosmetic Sheet Structures

The described invention provides cosmetic sheet structures for application to a region of skin of the human body. The cosmetic sheet structures of the described invention are compact and customizable, suitable for everyday use, and easily transportable for use on the go.

According to one aspect, the described invention features a cosmetic sheet structure for application to a region of skin of the human body, the cosmetic sheet structure comprising a cosmetic layer comprising material impregnated with at least one active ingredient, contained in the cosmetic sheet structure and transferrable to the region. According to some embodiments, the active ingredient is a cosmetic ingredient or a pharmaceutical ingredient. According to some embodiments, the active ingredient is selected from antioxidants, free-radical scavengers, moisturizers, depigmenting agents, liporegulators, anti-acne agents, anti-aging agents, cleansers, softeners, anti-wrinkle agents, keratolytic agents, anti-inflammatories, refreshening agents, UV absorbing agent, tanning agents, vascular protective agents, antibacterials, antifungals, antiperspirants, deodorants, antidandruff agents, skin conditioners, immunomodulators, essential oils and nourishing agents.

The cosmetic sheet structure is appropriate for application to any region of skin of the body. According to some embodiments, the cosmetic sheet structure is applied to a region of the skin on the face and/or neck. In other embodiments, the cosmetic sheet structure is applied to a region of the skin, excluding the face (for example, but not limited to, arms, legs, hands, chest, back, feet, etc.).

According to some embodiments, the skin is dry skin, including severely, moderately and mildly dry skin. According to some embodiments, the skin is chapped, or flaky skin.

In some embodiments, the skin is aged skin. As skin ages, it shows skin aging effects such as loss of elasticity, keratinization, formation of skin wrinkles and skin contraction. The cause of this skin gaining may be due to genetic factors or external factors such as UV and humidity, or a combination thereof.

According to some embodiments, the at least one active ingredient is formulated to form a film on the skin. According to some embodiments, the film is protective. According to some embodiments, the film is occlusive.

According to some embodiments, the cosmetic sheet structure further comprises a moisture retaining layer separably attached to the cosmetic layer, and retaining the moisture of the cosmetic layer when wearing the cosmetic layer on the face.

According to some embodiments, the at least one active ingredient is effective to penetrate the superficial layers of the skin, i.e. the epidermis. The epidermis comprises, from most superficial to deepest, the stratum corneum, stratum lucidium, stratum granulosm, stratum spinosum, stratum germinativum (also called stratum basale). According to some embodiments, the at least one active ingredient penetrates the stratum corneum. One example of a cosmetic effect within the stratum corneum is skin moisturization; which may involve the hydration of dry outer cells by surface films or the intercalation of water in the lipid-rich intercellular laminae. The stratum corneum also may serve as a reservoir phase or depot wherein topically applied substances accumulate due to partitioning into, or binding with, skin components.

According to some embodiments, the cosmetic sheet structures of the described invention are dermatologically-acceptable in that they do not have undue toxicity, incompatibility, instability, allergic response, and the like, when applied to skin. According to some embodiments, the cosmetic sheet structure is hypoallergenic.

According to some embodiments, the cosmetic sheet structures of the described invention can be pharmaceutically or cosmetically elegant. “Pharmaceutically elegant” and/or “cosmetically elegant” describes a composition that has particular tactile properties which feel pleasant on the skin (e.g., compositions that are not too watery or greasy, compositions that have a silky texture, compositions that are non-tacky or sticky, etc.). Pharmaceutically or cosmetically elegant can also relate to the creaminess or lubricity properties of the composition or to the moisture retaining properties of the composition.

The cosmetic sheet structures may be applied once a day, twice a day or more (e.g. 3, 4, 5, or more times a day). According to some embodiments, the cosmetic sheet structures are applied when the benefits of the cosmetic sheet structures are needed. According to some embodiments, the cosmetic sheet structures are applied decoratively.

Cosmetic Ingredients

According to some embodiments of the described invention, the sheet structure can contain at least one cosmetic ingredient.

The CTFA International Cosmetic Ingredient Dictionary and Handbook (2004 and 2008) describes a wide variety of non-limiting cosmetic ingredients that can be used in the context of the described invention. Examples of these ingredient classes include: fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraaminobenzoic acid (“PABA”) and corresponding PABA derivatives, titanium dioxide, zinc oxide, etc.), essential oils, vitamins (e.g. A, B, C, D, E, and K), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, and manitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate). Non-limiting examples of some of these ingredients are provided in the following subsections.

Dyes and Coloring Ingredients

According to some embodiments, the cosmetic sheet structure comprises a cosmetic ingredient, e.g, makeup, that can be applied directly to the skin. According to some embodiments, the cosmetic sheet structure enables the consumer to visualize the color, shade and texture of the makeup before application, as if it had been so applied. The consumer can then then compare the color and texture of the cosmetic sheet structures next to his/her facial coloring. The consumer can also compare cosmetic sheet structures with different colors. Further, by providing various shapes of cosmetic sheet structures, the attractive characteristics of the cosmetics are enhanced when the cosmetics are held in front of a facial area of the user. According to some embodiments, the cosmetic sheet structures comprising the cosmetic ingredient, e.g. makeup, may have an embossed, etched, glittery, shimmery or matte finish. According to some embodiments, the cosmetic ingredient is a blush. According to some embodiments, the cosmetic ingredient is a bronzer. According to some embodiments, the cosmetic ingredient is an eyeshadow. According to some embodiments, the cosmetic ingredient is a lip pigment. According to some embodiments, the cosmetic ingredient is a concealer. According to some embodiments, the cosmetic ingredient further comprises glitter.

Moisturizing Ingredients

Non-limiting examples of moisturizing agents that can be used with the compositions of the described invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, alanine, algae extract, aloe barbadensis, aloe-barbadensis extract, aloe barbadensis gel, althea officinalis extract, apricot (prunus armeniaca) kernel oil, arginine, arginine aspartate, arnica montana extract, aspartic acid, avocado (persea gratissima) oil, barrier sphingolipids, butyl alcohol, beeswax, behenyl alcohol, beta-sitosterol, birch (betula alba) bark extract, borage (borago officinalis) extract, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, eucalyptus globulus oil, evening primrose (oenothera biennis) oil, fatty acids, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, maltitol, matricaria (chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (oryza sativa) bran oil, RNA, rosemary (rosmarinus officinalis) oil, rose oil, safflower (carthamus tinctorius) oil, sage (salvia officinalis) oil, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

Anti-Aging Ingredients

According to some embodiments, the cosmetic ingredient is an anti-aging ingredient. According to some embodiments, the anti-aging ingredient delays, prevents, or treats signs of skin aging. According to some embodiments, the anti-aging ingredient delays, prevents, or treats wrinkles. According to some embodiments, the anti-aging ingredient delays, prevents, or treats fine lines. According to some embodiments, the anti-aging ingredient delays, prevents, or treats dryness. According to some embodiments, the anti-aging ingredient delays, prevents, or treats reduced firmness. According to some embodiments, the anti-aging ingredient delays, prevents, or treats reduced elasticity.

Exemplary anti-aging ingredients include retinol, retinyl esters, niacinamide, lecithin, adenosine, Coenzyme Q10, hormones, alpha hydroxy acids, caffeine, alpha-lipoic acid, salicylic acid, ceramides, lecithin, glycerin, fatty acids, polysaccharides, hyaluronic acid, sodium PCA, peptides, amino acids, cholesterol, glycosaminoglycans, vitamin A, vitamin C, vitamin E, superoxide dismutase, beta-carotene, glutathione, selenium, green tea and derivatives, and herbal extracts.

Vitamin C includes ascorbic acid, and also salts and esters thereof such as magnesium ascorbyl phosphate, ascorbyl palmitate, L-ascorbyl stearate, dehydroascorbic acid, Vitazyme C and combinations thereof. Vitamin A for purposes of this invention will include retinol, retinoic acid as well as retinyl C2-C22 fatty acid esters. Most preferred among the esters are retinyl palmitate and retinyl linoleate. Vitamin E may be provided in the form of tocotrienols, α-tocopherol, β-tocopherol, γ-tocopherol and δ-tocopherol. Included within the Vitamin E group are tocopheryl C2-C22 fatty acid esters including tocopheryl acetate, tocopherol linoleate and tocopheryl palmitate. Vitamin B may be present in the form of thiamine, riboflavin, niacin, pantothenic acid, biotin, cobalamin, pyridoxine hydrochloric, pyridoxamine dihydrochloride, pyridoxal, pyridoxal phosphate, folic acid, inositol and mixtures as well as complexes thereof. Under the term vitamin may also be included thaproline, L-caritine, nicotinic acid, nicotinamide and cyproterone acetate.

Herbal extracts particularly suitable for the described invention are antioxidants or free-radical inhibitors.

Whitening Ingredients

According to some embodiments, the cosmetic ingredient is a whitening agent. 4-methoxysalicylic acid is known to have a whitening effect. Selection of the salt of 4-methoxysalicylic acid is not limited in particular; examples include alkaline metal salts or alkaline earth metal salts such as a sodium salt, potassium salt, calcium salt, and magnesium salt, as well as an ammonium salt and amino acid salt. Other whitening agents include hydroquinone, vitamin C or a vitamin C derivative; sulfonyl methane (MSM), a naturally occurring sulfur compound; kojic acid; lactic acid and its derivatives such as D-lactic acid, L-lactic acid, DL-lactic acid, and their nonmetallic derivatives and lactates of alkali metal or alkaline earth metal; resveratrol and derivatives, for example an acetylated derivative of resveratrol or a hydroxy derivative thereof.

According to some embodiments, the skin whitening agent is selected for its ability to decrease melanin production by epidermal melanocytes.

UV Absorbing Ingredients

UV absorbing agents are capable of protecting skin from UV radiation (e.g., UVA, UVB, and/or UVC radiation). Non-limiting examples of UV Absorbing Agents that can be used in the context of the described invention include those listed in the International Cosmetic Ingredient Dictionary Handbook, 12th Edition (2008), Vol. 3, pages 3236-3239, which is incorporated by reference. Such examples include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (and octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, dibenzoylmethane derivatives (e.g., avobenzone), styrene/acrylates copolymer (e.g., SUNSPHERES™ sold by Rohm and Haas), octocrylene, etc. Non-limiting examples of physical sunblocks include, kaolin, talc, petrolatum and metal oxides (e.g., titanium dioxide and zinc oxide).

Antioxidants

The term “anti-oxidant agent” as used herein refers to a substance that inhibits oxidation or reactions promoted by oxygen or peroxides. Non-limiting examples of antioxidants that can be used with the compositions of the described invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

Structuring Agents

According to other non-limiting aspects, the compositions of the described invention can include a structuring agent. According to certain aspects, structuring agents assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents.

Essential oils typically are named by the plant from which the oil is found. For example, rose oil or peppermint oil are derived from rose or peppermint plants, respectively. Non-limiting examples of essential oils that can be used in the context of the described invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the described invention.

Preservatives

Non-limiting examples of preservatives that can be used in the context of the described invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

Pharmaceutical Ingredients

Pharmaceutical active agents are also contemplated as being useful with the cosmetic sheet structures of the described invention. Non-limiting examples of pharmaceutical active agents include anti-acne agents, agents used to treat rosacea, analgesics, anesthetics, anti-inflammatory antifungals, antimicrobials, antiperspirants, antipruritics, antipsoriatic agents, antiseborrheic agents, biologically active proteins and peptides, burn treatment agents, cauterizing agents, depigmenting agents, depilatories, enzymes, hair growth stimulants, hair growth retardants, cold sore treatment agents, photosensitizing actives, skinprotectant/barrier agents, steroids including hormones and corticosteroids, sunburn treatment agents, transdermal actives, vasoconstrictors, wound treatment agents, wound healing agents, etc.

According to some embodiments, the pharmaceutical ingredient is an anti-inflammatory agent. Anti-inflammatories of the described invention are illustrated by corticoids such as beta-methasone 17-acetate, indomethacin, ketoprofen, flufenamic acid, ibuprofen, diclofenace, diflunisal, fenclofenac, naproxen, piroxidam and sulindac. Antimicrobials illustrative of the described invention include, chlorohexidine, hexetidine, 3,4,4′-trichlorocarbanilide, (tricarbanilide) 2,4,4′-trichloro-2-hydroxydiphenyl ether (triclosan), cetyl pyridinium chloride, benzalkonium chloride, C2-C20 organoperoxy compounds (e.g. benzoyl peroxide) and mixtures.

According to some embodiments, the pharmaceutical ingredient is a vasoconstrictor. Vasoconstrictors are illustrated by compounds such as papaverine, yohimbine, visnadin, khellin, bebellin and nicotinate derivatives. Zinc salts which may be effective include zinc thaproline, zinc chloride, zinc sulfate, zinc phenolsulfonate and zinc pyrithione. Other substances within one or more of the above categories of actives include resorcinol, azelaic acid, oxamic acid and cyoctol.

Anti-Microbials

The disclosed cosmetic sheet structures of the described invention may also include various ingredients to prevent the action of microorganisms. The prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The impregnation amount of the at least one active ingredient to be impregnated into the cosmetic sheet structure is not limited in particular; it is adjusted appropriately according to the characteristics of the sheet and the active agent(s). According to some embodiments, the amount of impregnation of the active ingredient to be impregnated into the sheet of the said sheet-like cosmetic is preferably 5-15 times of the mass of the sheet having the non-woven fabric laminated structure.

The cosmetic sheet structure of the described invention can include any number of combinations of cosmetic ingredients described throughout this specification. The concentrations of any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein.

According to non-limiting aspects, the percentage can be calculated by weight or volume of the total composition. A person of ordinary skill in the art would understand that the concentrations can vary depending on the addition, substitution, and/or subtraction of ingredients in a given composition. In percent solutions, the amount (weight or volume) of a solute is expressed as a percentage of the total solution weight or volume. Percent solutions can take the form of weight/volume % (wt/vol % or w/v %), weight/weight % (wt/wt % or w/w %), or volume/volume % (vol/vol % or v/v %). In each case, the percentage concentration is calculated as the fraction of the weight or volume of the solute related to the total weight or volume of the solution.

Backing

According to some embodiments, the cosmetic sheet structure further comprises a backing. The backing can be attached to the cosmetic layer by moisture and have a form capable of being separated from the cosmetic layer.

According to some embodiments, the backing comprises a design print layer that makes an aesthetic design that may be integrally formed on the backing. According to some embodiments, the backing is colored by colored particles dispersed therein. According to According to some embodiment some embodiments, the backing may be any size or shape.

According to some embodiments, the backing may include an ultraviolet blocking agent so as to have an ultraviolet blocking property.

Non-Woven Matrix

According to some embodiments, the cosmetic sheet structure comprises a non-woven matrix. A non-woven matrix may be prepared by air-laying or water-laying processes in which the fibers or filaments are first cut to the desired length from long strands, passed into water or airstreams, and then deposited onto a screen or mesh through which the fiber-laid in air or water is passed. The resulting non-woven layer, regardless of its method or production or composition, is then subjected to at least one of several types of bonding operations to anchor the individual fibers together to form a self-sustaining web. Specific processes for preparing the non-woven substrate include hydroentanglement, thermal bonding and combinations of these processes.

Nanofiber Web

According to some embodiments, the cosmetic sheet structure comprises a nanofiber web obtained by electrospinning a water-soluble polymer and the active ingredient together. Nanofibers can provide skin contact areas much larger than conventional nonwoven fabrics, and can be spun at room temperature and atmospheric pressure. Accordingly, various functional materials are mixed with a spinning solution, and the spinning solution mixed with the various functional materials is spun, to then be manufactured in the form of nanofibers mounted with the functional materials at the same time of spinning.

Accordingly, one object of the described invention is to provide a cosmetic sheet in which an active ingredient, for example a cosmetic ingredient or pharmaceutical ingredient, is melted by moisture and absorbed into the skin, by using a nanofiber web obtained by electrospinning a water-soluble polymer and a functional material together. Another one object of the described invention is to provide a cosmetic sheet in which an active ingredient is melted by heat and absorbed into the skin, by using a nanofiber web obtained by electrospinning a water-soluble polymer and a functional material together.

According to some embodiments, the cosmetic layer is prepared by electrospinning a spinning solution in which a water-soluble polymer material, an active ingredient and a crosslinking agent are dissolved together in a solvent to form nanofibers, and accumulating the nanofibers to form; when adhering the nanofibers to the skin, the nanofiber webs are slowly dissolved by moisture and absorbed into the skin while being removed. According to some embodiments, the nanofibers have a diameter of less than 1 μm. According to some embodiments, the nanofiber webs have micropores in which a basis weight of the nanofibers is set in the range of 10 to 50 gsm, i.e., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 gsm (grams per square meter).

The solvent can be water or alcohol, or a mixed solvent thereof.

The cosmetic layer is prepared by dissolving a hydrophilic polymer and a functional material in a solvent to prepare a spinning solution, then electrospinning the spinning solution to form nanofibers having a fiber diameter of less than 1 μm, and accumulating the nanofibers, in the form of a nanofiber web having micropores.

The spinning methods that can be used are known in the art, and can be any one of general electro spinning, air-electro spinning (AES), electrospray, electrobrown spinning, centrifugal electro spinning, and flash-electro spinning.

The cosmetic layer can be spun by blending the water-soluble polymer alone or in combination of two or more kinds of the water-soluble polymers. The solvent may employ water, alcohol, or the like having compatibility with the polymer to then be used, alone or in combination thereof.

When preparing the cosmetic layer, a crosslinking agent is mixed with the spinning solution for crosslinking the water-soluble polymer. For example, according to some embodiments, when dissolving a polymer material, spinning solution can be prepared while heating and stirring the polymer material, cooling the spinning solution to room temperature, and then adding a crosslinking agent so that the crosslinking agent is added to the spinning solution in such a range that a crosslinking reaction does not occur.

Therefore, according to some embodiments, the cosmetic layer of the described invention can be prepared so that the addition of the crosslinking agent during the preparation of the spinning solution is performed in the final step after dissolving the water-soluble polymer in water or alcohol and adding the functional material in the dissolved result. According to some such embodiments, the content of the crosslinking agent to be added, for example, is in the range of 0.1 wt % to 2 wt % (i.e., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2 wt %), with respect to the polymer. The crosslinking can be performed by a method such as hot air blowing, heat treatment calendering, UV irradiation, etc. The content and time of the crosslinking agent can be adjusted according to the purpose of use.

The water-soluble polymer material can be electrospun and can include synthetic polymers or natural polymers. The synthetic polymers or natural polymers can be used alone or in combination thereof, but any polymers that can be dissolved in water or alcohol to form nanofibers by electrospinning are not particularly limited.

Examples of such water-soluble polymer materials include, without limitation, one selected from polyvinyl alcohol (PVA), polyvinyl pyrrolidone (PVP), polyethylene oxide (PEO), carboxyl methyl cellulose (CMC), starch, polyacrylic acid (PAA), and hyaluronic acid, or a mixture of two or more thereof.

Examples of active ingredients, for example a cosmetic ingredient or pharmaceutical ingredient, are described herein.

In addition, all the components used in the cosmetic sheet for the purposes of oxidation prevention, skin and hair conditioning, emulsion stability, pH control, antibacterial effects, and the like can be constituted alone or as a mixture thereof.

According to some embodiments, the active ingredient is added in an amount of about 0.5 wt % to about 50 wt % (i.e., 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 15.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 47.0, 47.5, 48.0, 48.5, 49.0, 49.5, or 50 wt %) relative to the water-soluble polymer.

The crosslinking agent or co-crosslinking agent can be formed of at least one selected from para-toluene sulfonic acid (TSA), polyurea-co-formaldehyde, tri-methylpropane trimethacrylate (TMPTMA), divinylbenzene (DVB), N-(1-hydroxy-2,2-dimethoxyethyl) acrylamide, N, N.-methylenebisacrylamide, ethylene glycol diacrylate, di (ethylene glycol) diacrylate, boric acid, and glutaraldehyde.

According to s some embodiments, the crosslinking density of the water-soluble polymer is altered in order to control the delivery rate at which an effective ingredient is delivered to the skin according to properties of the effective ingredient, and a method of manufacturing the same. Thus, the cosmetic sheet according to the described invention may control a dissolving rate at which a water-soluble polymer is melted by moisture or heat, by controlling a crosslinking density of the water-soluble polymer thereby controlling a delivery rate at which an active ingredient is delivered to the skin according to properties of the active ingredient.

A release film may be detachably attached to the cosmetic layer when the cosmetic sheet is attached to the skin, and a separation knob for easily separating the cosmetic sheet or the release film from each other may be formed in the cosmetic sheet or the release film.

The moisture retaining layer may be provided with a separating knob for easily separating the moisture retaining layer and the cosmetic layer from each other.

Manufacturing processes of the cosmetic sheet are described infra.

Physical Properties

According to some embodiments, the cosmetic sheet structure is flexible (meaning capable of being bent, usually without breaking). Such flexibility is useful, e.g., in conforming to various shapes of the face, body, etc. According to some embodiments, the shape is formed to match the area where it is applied.

According to some embodiments, the cosmetic sheet structure forms a tape (meaning a narrow strip of material) for application to the skin. According to some embodiments, the cosmetic sheet structure forms a shape for application to the skin. The shape may be selected from square (with or without rounded edges), rectangular (with or without rounded edges), circular, an oval, elliptical, kidney shaped, diamond, heart, or star. The cosmetic sheet structure may also be formed in a customized shape, specified by the user.

### III. Application

The cosmetic sheet according to the described invention is melted and absorbed into the skin on contact with water or an aqueous solution (e.g. moisture or mist). Thus, according to some embodiments, the cosmetic sheet structure according to the described invention is attached to the skin in the form of a thin film type nanofiber web and has a dissolution feature, to thus advantageously enable a wearer who uses the cosmetic sheet structure closely adhered to the skin to do daily activities.

According to some embodiments, the cosmetic sheet according to the described invention may control a dissolving rate at which a water-soluble polymer is melted by moisture or mist, by controlling a crosslinking density of the water-soluble polymer, thereby controlling a delivery rate at which an active ingredient is delivered to the skin. Accordingly, the at least one active ingredient is transferred to the region when the cosmetic sheet structure is wetted by water or an aqueous solution.

The cosmetic sheet according to the described invention may be melted by heat. Accordingly, the at least one active ingredient is transferred to the region when the cosmetic sheet structure is heated by touch.

According to some embodiments, the cosmetic sheet structure is transferrable from one region of the skin to another region of the skin.

### IV. Methods of Use

According to certain aspects of the described invention, the cosmetic sheet structures improve the visual appearance of their skin. For instance, symptoms associated with dry skin (e.g., flaky skin, dried or rough tactile quality, cracked skin, dehydrated skin, itchy skin, or red or erythemic skin) are associated with unattractive skin. Further, effects of aging (wrinkles, fine lines, age spots) are associated with unattractive skin. The cosmetic sheet structures of the described invention are used to improve any one or more of these symptoms, as well as for decorative purposes.

Thus, the described invention features a method of improving the appearance of the skin, the method comprising contacting one or more regions of the skin with one or more cosmetic sheet structures as described herein, thereby improving the appearance of the skin.

According to some embodiments, an improvement in the appearance of the skin comprises an improvement in one or more of wrinkles, dryness, discoloration, imperfection, scarring, redness, radiance, softness or blemishing of the skin.

According to another aspect, the described invention features a method of protecting the skin from external damage, the method comprising contacting on or more regions of the skin with one or more cosmetic sheet structures as described herein, thereby protecting the skin from external damage. According to some embodiments, the external damage is caused by ultraviolet radiation. According to some embodiments, the external damage is caused by temperature.

According to another aspect, the described invention features a method of applying a cosmetic composition to the skin, the method comprising contacting one or more regions of the skin with one or more cosmetic sheet structures as described herein, thereby protecting the skin from external damage. According to some embodiments, the cosmetic composition comprises a pigment. According to some embodiments, the cosmetic composition is selected from the group consisting of: blush, bronzer, eyeshadow, lip pigment and concealer.

The one or more cosmetic sheet structures may be applied to the same area of the skin, or applied to multiple different areas of the skin. According to some embodiments, the cosmetic sheet structures are applied to one area of the skin and transferred to another area of the skin.

The cosmetic sheet structures are activated by water or an aqueous solution or are activated by heat, as described herein. According to some embodiments, the one or more cosmetic sheets dissolve after activation. According to some embodiments, cosmetic sheets dissolve about 30 seconds to 1 hour after activation, for example 30 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes or more after activation.

According to some embodiments, the one or more cosmetic sheet structures are applied for a time of about 1 minute to 5 hours, for example 1 minute, 5 minutes, 10 minutes, 15 minutes, 30 minutes, 45 minutes, 60 minutes, 2 hours, 3 hours, 4 hours or 5 hours or more.

According to other embodiments, the cosmetic sheets do not leave a residue on the area of the skin after dissolving. According to some embodiments, once the product is applied and activated (e.g. with water or an aqueous solution or with heat), only then it dissolves and spread throughout the skin area living no residue.

Also disclosed is a method of treating or preventing a skin condition comprising topical application of any one of the compositions described in this specification to skin in need thereof, wherein the topical application of the composition treats the skin condition. According to one aspect, the method includes moisturizing skin or treating or preventing the appearing of dry skin, flaky skin, or chapped skin. In other aspects, it is contemplated that the cosmetic sheets described herein can be used to treat or prevent skin conditions ranging from pruritus, spider veins, lentigo, age spots, senile purpura, keratosis, melasma, blotches, fine lines or wrinkles, nodules, sun damaged skin, dermatitis (including, but not limited to seborrheic dermatitis, nummular dermatitis, contact dermatitis, atopic dermatitis, exfoliative dermatitis, perioral dermatitis, and stasis dermatitis), psoriasis, folliculitis, rosacea, acne, impetigo, erysipelas, erythrasma, eczema, and other inflammatory skin conditions. According to certain non-limiting aspects, the skin condition can be caused by exposure to UV light, age, irradiation, chronic sun exposure, environmental pollutants, air pollution, wind, cold, heat, chemicals, disease pathologies, smoking, or lack of nutrition. The skin can be facial skin or non-facial skin (e.g., arms, legs, hands, chest, back, feet, etc.).

The method can further comprise identifying a person in need of skin treatment. The person can be a male or female. The age of the person can be at least 3 months, 4 months, 5 months, 6 months, 7 months, 8 months, 9 months, 10 months, 11 months, 1, 2, 3, 4, 5, 6, 7, 8, 9, 10, 15, 20, 25, 30, 35, 40, 45, 50, 55, 60, 65, 70, 75, 80, 85, 90, 95, or more years old, or any range derivable therein. The method can also include topically applying a cosmetic sheet comprising an active agent in an amount effective to: increase the stratum corneum turnover rate of the skin; increase collagen synthesis in fibroblasts; increase cellular anti-oxidant defense mechanisms (e.g., exogenous additions of anti-oxidants can bolster, replenish, or prevent the loss of cellular antioxidants such as catalase and glutathione in skin cells (e.g., keratinocytes, melanocytes, langerhans cells, etc.) which will reduce or prevent oxidative damage to the skin, cellular, proteins, and lipids); inhibit melanin production in melanocytes; reduce or prevent oxidative damage to skin (including reducing the amount lipid peroxides and/or protein oxidation in the skin). According to some embodiments, compositions of the described invention can decrease the amount of internal oxidation and/or external oxidative damage in a cell. In other aspects, the compositions can increase collagen synthesis in a cell. The cosmetic sheets described herein can also reduce skin inflammation, such as by reducing inflammatory cytokine production in a cell. Non-limiting examples of such cells include human epidermal keratinocyte, human fibroblast dermal cell, human melanocytes, three dimensional human cell-derived in vitro tissue equivalents comprising human keratinocytes, human fibroblasts, or human melanocytes, or any combination thereof (e.g., combination of human keratinocytes and human fibroblasts or a combination of human keratinocytes and human melanocytes).

### V. Methods of Making

According to some embodiments of the described invention, the cosmetic sheet structure is made as follows:

### Preparation of Spinning Solution Containing Hydrophilic Polymer and Cosmetic Ingredient

According to some embodiments, a spinning solution is prepared to form a cosmetic layer by dissolving a hydrophilic polymer at a spinnable concentration in a solvent such as water or alcohol. The concentration of the spinning solution is suitable when maintaining the fibrous morphology during spinning, and the polymer material in the spinning solution (solvent+polymer material) is suitable in the range of about 5 to 70 wt % (5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt %).

In the case where the proportion of the polymer is less than 5 wt %, a drop due to a low concentration is formed rather than forming nanofibers during electrospinning, so that fibers cannot be formed in many cases. Since the content of the polymer is too much when the amount is more than 70 wt %, it is not possible to form nanofibers due to poor spinning. Therefore, it is necessary to prepare a spinning solution in a suitable concentration range in which fibers can be formed depending on the kind of polymer used. In particular, when two or more polymers are blended and spun, the polymer and solvent should be compatible with each other and should be satisfied with conditions that do not cause phase separation and the like. In addition, it is preferable to prepare a spinning solution by mixing one or two kinds of solvents while considering the volatilization of the solvent.

In addition, when preparing a spinning solution by mixing a cosmetic material and a crosslinking agent, it is necessary to prepare the spinning solution by dissolving the polymer material and injecting the cosmetic material and the crosslinking agent at room temperature. When PVA or the like is used as a polymer, heating and stirring are generally carried out in the preparation of a spinning solution. Therefore, when a crosslinking agent is added, a crosslinking reaction may occur and a solid component may be formed without forming a solution.

### Formation of Cosmetic Layer

An electrospinning device includes a spinning nozzle for electrospinning a spinning solution, and a collector for accumulating microfine fiber yarns spun from the spinning nozzle to sequentially form a cosmetic layer and a moisture retaining layer.

In this state, a voltage is applied to the spinning nozzle to conduct electrospinning, by using a high voltage generator. The use voltage is a voltage capable of being spun in the range of 2 kV-100 kV.

For example, a discharge amount during spinning can be discharged and spun in the range of 0.01 to 5 cc/holemin, and the spinning performed in an environment of a relative humidity of 10 to 90% in a chamber capable of controlling temperature and humidity during spinning. In order to improve handling properties, the basis weight of the nanofiber web can be in the range of 10-50 gsm.

The spinning solution is made into ultrafine fiber strands in the spinning nozzle and is spun onto the surface of the release film 40. Ultrafine nanofibers are then accumulated on the surface of the release film 40 to form a cosmetic layer 10 in the form of a nanofiber web having a plurality of pores.

### Crosslinking and Thermocompression (Calendering)

In order to control the time that the electrospun nanofiber web is melted by moisture or mist, it is necessary to perform a crosslinking process and a thermocompression process to increase a bonding force between the nanofibers.

The crosslinking is controlled so that the complete crosslinking or the partial crosslinking proceeds to be self-melt over time by moisture, mist, or the like. The crosslinking can be carried out before or after thermo compression or calendering, or can proceed simultaneously with calendering. For partial crosslinking, the type and content of the crosslinking agent in the spinning solution and the time of hot air, heat treatment calendering, and UV irradiation can be adjusted.

In this case, partial crosslinking can be carried out in the range that the deterioration or destruction of the functional material does not occur, within the range of 80−100° C. for 30 minutes or less, but in the case of the heating and calendering at 150° C., partial crosslinking is carried out for 30 seconds or less, and can be executed in the range that the deterioration of the functional material and the partial crosslinking proceeds.

### Lamination of Moisture Retaining Layer

According to some embodiments, the cosmetic sheet structure further comprises a moisture retaining layer. For example, a moisture retaining layer is laminated on one side of the cosmetic layer. The moisture retaining layer is formed of a polyurethane film and attached to one surface of the cosmetic layer by moisture contained in the cosmetic layer.

In this case, a polyurethane film comprising a design pattern layer that can create an aesthetic design may be integrally formed on one surface of the moisture retaining layer.

As described above, since the cosmetic layer and the moisture retaining layer are attached to each other by moisture, they can be separated easily and conveniently, and when the cosmetic layer is melted by moisture and disappears, the moisture retaining layer can be separated from the face naturally.

### VI. Packaging

According to some embodiments, the cosmetic sheet structure is packaged in a protective cover or pouch. For example, pouches of the described invention can be of the laminated foil variety. These are heat sealed and utilize foils with very low vapor (e.g. moisture) transmission rates (a rate of transmission less than 5% per day, e.g., less than 1% per day volatile fluid gain or loss). Walls suitable for the pouch may utilize polyester, polyethylene or polypropylene sheets, several layers of which can be laminated together. These layers may also be provided with a coating of wax or other volatile fluid impermeable material.

According to some embodiments, the cosmetic sheet structure is contained within a dispensing unit. The dispensing unit can comprise a jagged edge to tear the sheet. According to some embodiments, the dispensing unit further comprises a second cosmetic sheet structure contained within a second dispensing unit. According to some embodiments, the dispensing units are connected. For example, the dispensing units can be connected by one or more magnets.

According to other embodiments, the one or more cosmetic sheet structures are contained within a housing unit. For example, the housing unit can further comprisesa mirror. According to some embodiments, the one or more cosmetic sheet structures are contained within one or more housing units. According to some embodiments, the housing units are connected. For example, the housing units can be connected by one or more magnets.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any disclosure disclosed or claimed herein or that it alone, or in any combination with any other reference or references, teaches, suggests or discloses any such disclosure. Further, to the extent that any meaning or definition of a term in this document conflicts with any meaning or definition of the same term in a document incorporated by reference, the meaning or definition assigned to that term in this document shall govern.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, exemplary methods and materials have been described.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Cosmetic Sheet Structure Activated by Water or Heat

This example describes a cosmetic sheet structure that is applied to the skin, preferably facial skin, and that is activated by water, for example moisture or mist. The exemplary cosmetic sheet structure comprises a cosmetic layer comprising material impregnated with at least active ingredient contained in the cosmetic sheet structure and transferrable to the region.

The exemplary cosmetic sheet structure comprises a backing and a material impregnated with a cosmetically effective formulation. The backing is any size, shape or color. For example, the backing is in a shape which fits underneath the eye to provide any one or more of moisturization, anti-aging, anti-wrinkle, smoothing, or brightening benefits to the under eye area. The active ingredient forms a film on the skin, providing, e.g., a moisturizing, anti-aging, smoothness, or brightening effect. For example, an effect within the stratum corneum is skin moisturization; which may involve the hydration of dry outer cells by surface films or the intercalation of water in the lipid-rich intercellular laminae; the stratum corneum also may serve as a reservoir phase or depot wherein topically applied substances accumulate due to partitioning into, or binding with, skin components. Thus, the exemplary product is effective to penetrate superficial layers of the skin.

The force needed to apply the cosmetic sheet structure to the skin is very low, for example the force needed to apply a wet tissue to the skin.

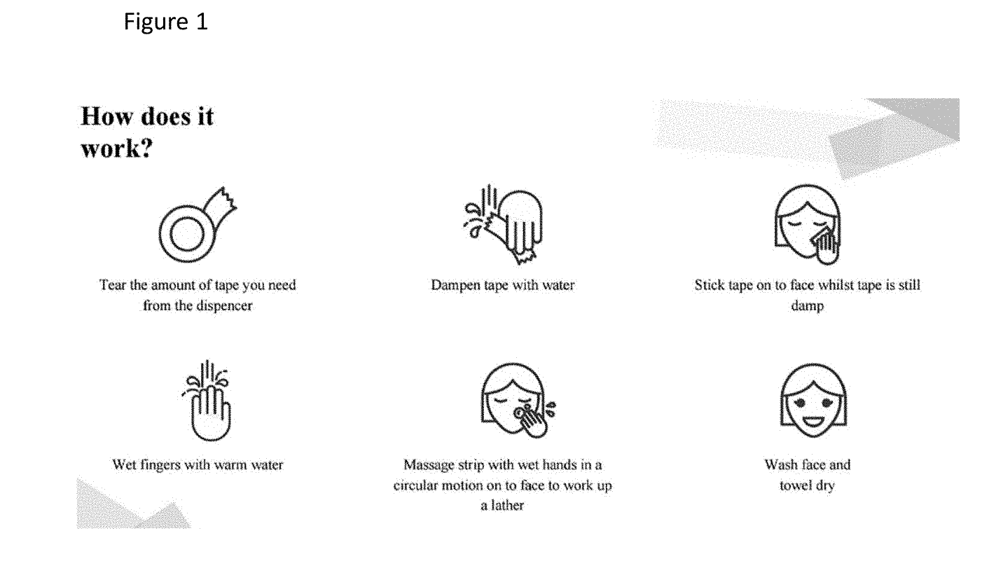

Once applied to the skin the cosmetic sheet structure dissolves, either by application of water (e.g. moisture or mist) or by heat (e.g. by touch). Once the product is applied and activated with very little water, only then it dissolves spread through out the skin area living no residue, as shown in FIG. 1.

The cosmetic sheet structures are made by the methods described herein.

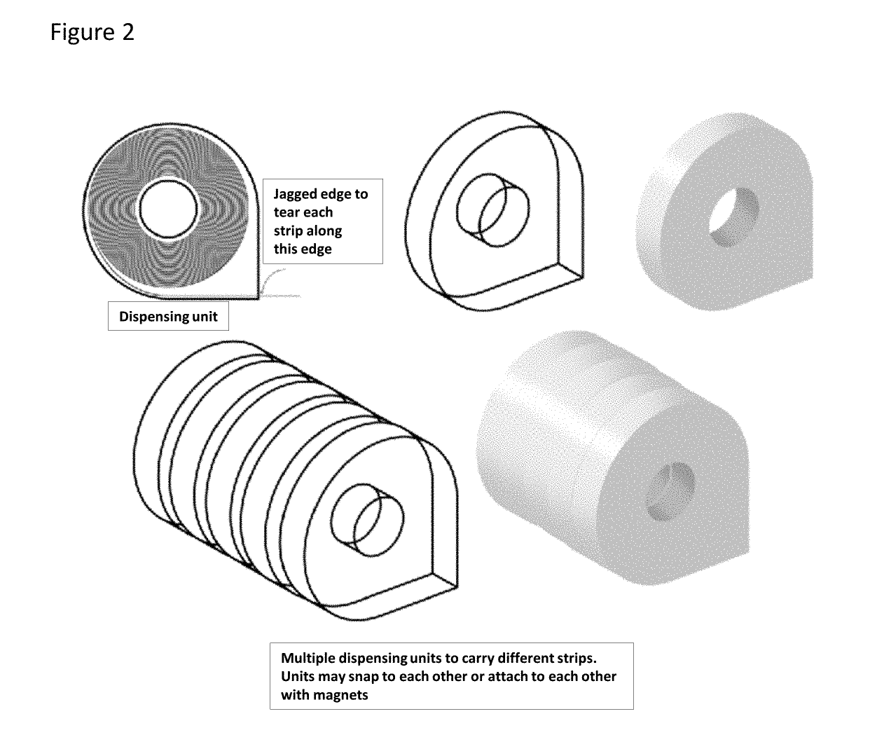

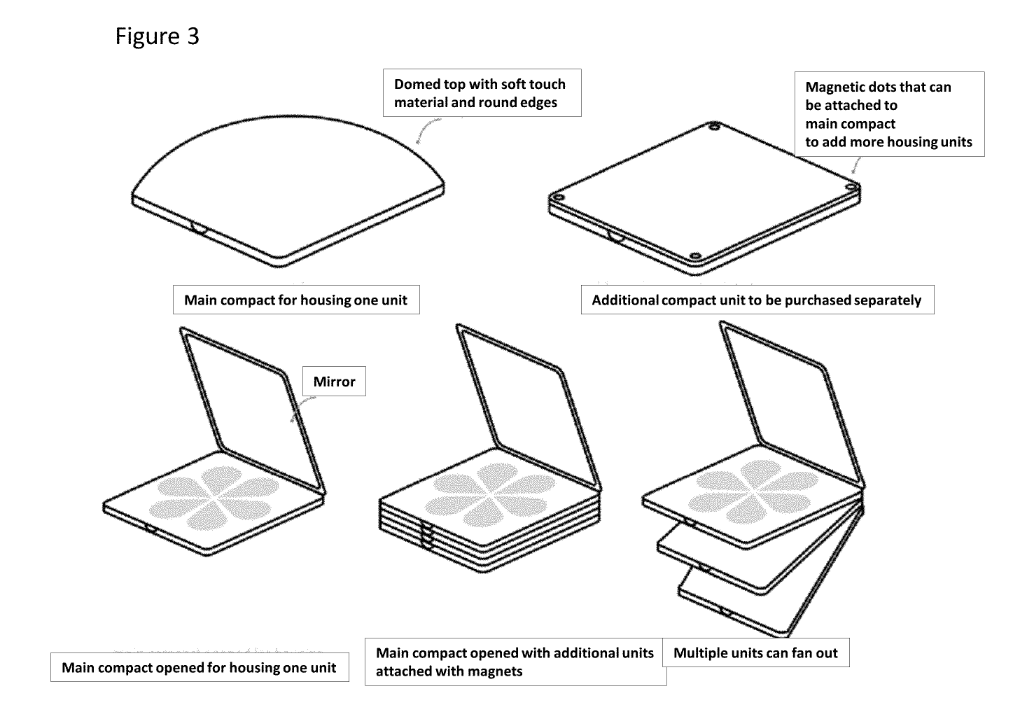

The cosmetic sheet structures may be housed in various dispensing units, either alone or in a combination of units combined together. The housing of the cosmetic sheet structures provides easy transport of the cosmetic sheet structures. FIG. 2 is a schematic of a dispensing unit for the cosmetic sheet structures. The dispensing unit may include a jagged edge to tear each strip along the edge of the dispensing unit. Multiple dispensing units are used to carry different strips, wherein the units may snap to each other or attach to each other with magnets. FIG. 3 is a schematic of another exemplary housing unit that has a domed to with soft touch material and round edges. An additional housing unit comprising the cosmetic sheet structures may be purchased separately, and then connected to the first housing unit by, e.g., magnets. The multiple units may fan out, and one or more of the units may comprise a mirror.

### Example 2. Face Caviar

According to other aspects, the described invention features a facial moisturizer comprising concentrated active ingredients as described herein, referred to herein as “face caviar.” According to some embodiments the face caviar is comprised within a pearl or a pod structure. Each pearl or pod is filled, with concentrated active ingredients that are freshly sealed into each pod, and the active ingredients are released only when needed. Accordingly, the user may use only what is needed, and there is no waste. According to some embodiments, each pearl or pod comprises an anti-aging ingredient as described herein.

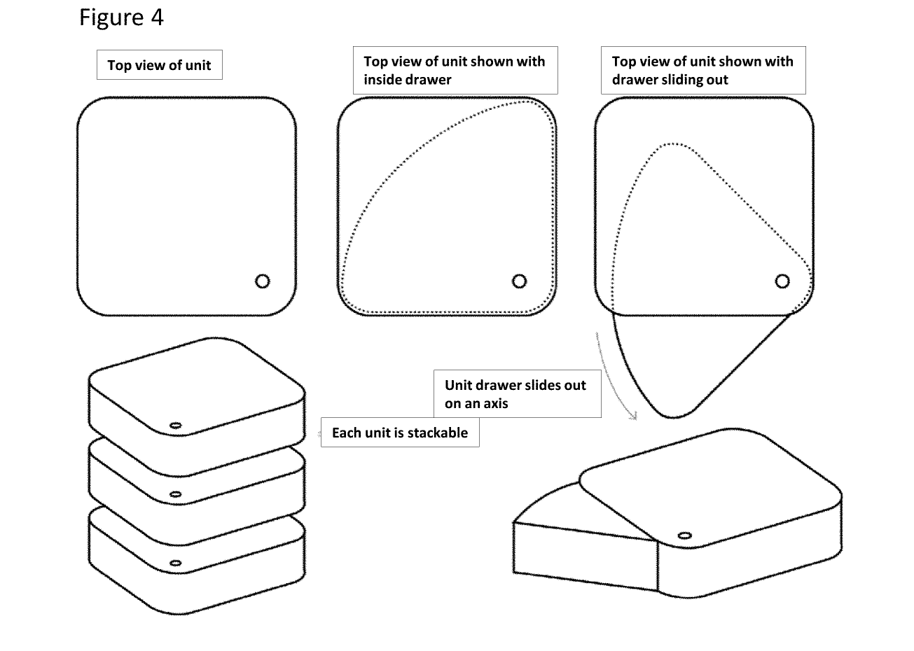

According to some embodiments, one or more pearls or pods are contained within a housing unit, as shown in FIG. 4. For example, 5, 10, 15, 20 or more pearls or pods may be contained in a housing unit. According to exemplary embodiments, the housing unit further comprises a mirror. According to some embodiments, the one or more pearls or pods are contained within one or more housing units, as shown in FIG. 4. According to some embodiments, the housing units are connected. For example, the housing units can be connected by one or more magnets.

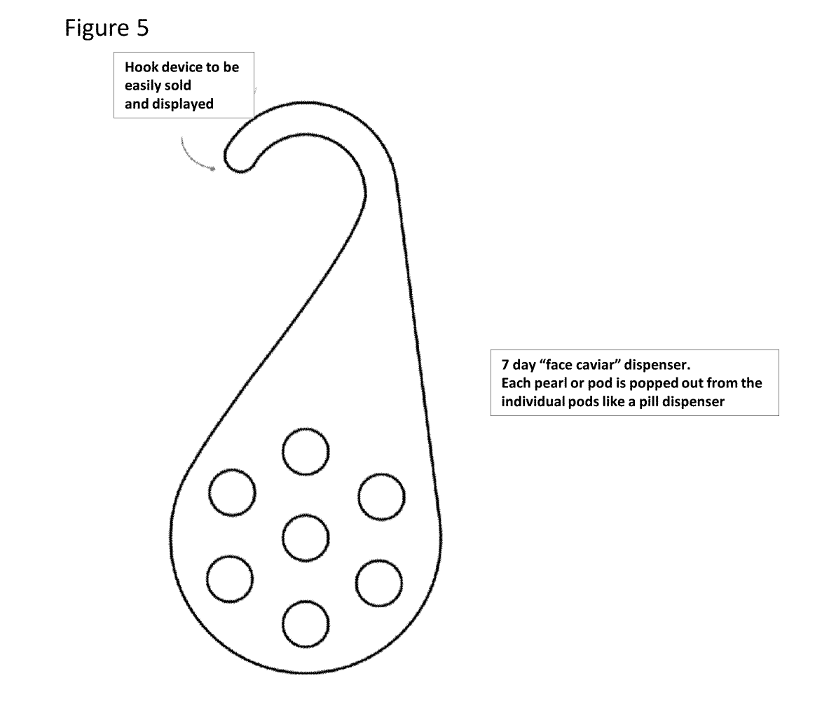

According to some embodiments, the pearls or pods are contained in a housing unit, for example as shown in FIG. 5. The housing unit comprises, for example, one pearl or pod for every day in a week of administration. FIG. 5 shows a 7 day “face caviar” dispenser. Each pearl or pod is popped out like a pill dispenser. As shown in FIG. 6, the housing unit may further comprise a hook device to be easily sold and displayed.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

What is claimed is:

1. A cosmetic sheet structure for application to a region of skin of the human body, the cosmetic sheet structure comprising:
a cosmetic layer comprising material impregnated with at least one active ingredient contained in the cosmetic sheet structure and transferrable to the region.

2. The cosmetic sheet structure of claim 1, further comprising a backing.

3. The cosmetic sheet structure of claim 1, wherein the cosmetic sheet structure is applied to a face, a neck, or both.

4. The cosmetic sheet structure of claim 1, wherein the active ingredient is a cosmetic ingredient or a pharmaceutical ingredient.

5. The cosmetic sheet structure of claim 1, wherein the skin is aged skin or dry skin.

6. The cosmetic sheet structure of claim 1, wherein the at least one active ingredient is formulated to form a film on the skin.

7. The cosmetic sheet structure of claim 1, wherein the active ingredient is one or more selected from the group consisting of: an antioxidant, a free-radical scavenger, a moisturizer, a depigmenting agent, a liporegulator, an anti-acne agent, an anti-aging agent, a cleanser, a softener, an anti-wrinkle agent, a keratolytic agent, an anti-inflammatory agent, a refreshening agent, a cicatrizing agent, a tanning agent, a vascular protective agent, an antibacterial agent, an antifungal agent, an antiperspirant agent, a deodorant agent, an antidandruff agent, a skin conditioner, an immunomodulator agent, and a nourishing agent.

8. The cosmetic sheet structure of claim 1, wherein the active ingredient is selected from the group consisting of: a blush, a bronzer, an eyeshadow, a lip pigment and a concealer.

9. The cosmetic sheet structure of claim 1, wherein the active ingredient is transferred to the region when the cosmetic sheet structure is heated by touch or wherein the active ingredient is transferred to the region when the cosmetic sheet structure is wetted by water or an aqueous solution.

10. The cosmetic sheet structure of claim 1, wherein the active ingredient is effective to penetrate the superficial layers of the skin.

11. The cosmetic sheet structure of claim 1, wherein the cosmetic sheet structure is flexible.

12. The cosmetic sheet structure of claim 1, wherein the cosmetic sheet structure is transferrable from one region of the skin to another region of the skin.

13. The cosmetic sheet structure of claim 1, wherein the cosmetic sheet structure is in form of a tape for application to the skin.

14. The cosmetic sheet structure of claim 1, wherein the cosmetic sheet structure is in form of a shape for application to the skin.

15. The cosmetic sheet structure of claim 1, wherein thickness of the cosmetic sheet structure is between about 0.01 to 1.0 centimeters.

16. The cosmetic sheet structure of claim 1, wherein the cosmetic sheet structure further comprises one or more ingredients selected from the group consisting of: a whitening agent, an ultraviolet absorbent, a surfactant, a humectant, a thickener, an alcohol, a powder ingredient, a coloring agent, a pH adjusting agent, a stabilizer, a preservative, a perfume, water, glitter, and a skin nutrient.

17. The cosmetic sheet structure of claim 1, wherein the cosmetic sheet structure comprises a non-woven matrix.

18. The cosmetic sheet structure of claim 1, wherein the cosmetic sheet structure comprises a nanofiber web obtained by electrospinning a water-soluble polymer and the cosmetic ingredient together.

19. The cosmetic sheet structure of claim 1, wherein the cosmetic sheet structure is hypoallergenic.

20. The cosmetic sheet structure of claim 1, wherein the cosmetic sheet structure is packaged in a protective cover or contained within a dispensing unit.

21. The cosmetic sheet structure of claim 1, wherein the one or more cosmetic sheet structures are contained within one or more housing units.

22. A method of improving the appearance of skin, the method comprising contacting one or more regions of the skin with one or more cosmetic sheet structures of claim 1, thereby improving the appearance of the skin.

23. A method of protecting skin from external damage, the method comprising contacting on or more regions of the skin with one or more cosmetic sheet structures of claim 1, thereby protecting the skin from external damage.

24. The method of claim 23, further comprising activating the cosmetic sheet structure with an aqueous solution or heat, wherein the one or more cosmetic sheets dissolve after activating.

25. The method of claim 24, wherein the cosmetic sheets do not leave a residue on the area of the skin after dissolving.
